(19) United States
(12) Patent Application Publication
(10) Pub. No.: US 2024/0260897 A1
(43) Pub. Date: Aug. 8, 2024

(54) Systems and Methods for Training Muscle Activation and Awareness

(71) Applicant: Lisa Sibits, Broadview Heights, OH (US)

(72) Inventor: Lisa Sibits, Broadview Heights, OH (US)

(21) Appl. No.: 18/104,792

(22) Filed: Feb. 2, 2023

(51) Int. Cl. A61B 5/00; A61B 5/11

(52) U.S. Cl. CPC A61B 5/486; A61B 5/1116; A61B 5/6804; A61B 5/6833; A61B 5/746; A61B 2562/166

(57) ABSTRACT

A device designed to improve the activation and awareness of at least one region of the body (e.g., the core abdominal muscles with emphasis on the transverse abdominis, and one or more other regions of the body) in appropriate postures. In particular, this disclosure relates to a device worn by the user that provides a means to alert the user to activate at least one region of the body (e.g., to activate the core abdominal muscles with emphasis on the transverse abdominis) in appropriate postures.

### CROSS-REFERENCE TO RELATED APPLICATIONS

This application claims priority to Provisional Patent Application No. 63/267,445, entitled AB ZIPPER CORE ABDOMINAL RETRAINER, filed Feb. 2, 2022, the entirety of which is incorporated herein by reference.

### FIELD OF THE INVENTION

This disclosure relates to a wearable device designed to improve the activation and awareness of at least one region of the body (e.g., the core abdominal muscles with emphasis on the transverse abdominis, and one or more other regions of the body) in appropriate postures. In particular, this disclosure relates to a device worn by the user that provides a means to alert the user to activate at least one region of the body (e.g., to activate the core abdominal muscles with emphasis on the transverse abdominis) in appropriate postures.

### BACKGROUND OF THE INVENTION

The core muscles work together to provide stability, posture support, align the body to resist forces, coordinate movement between the spine and extremities, and provide stability for the extremities to operate off of. In the adult population, there is often a lack of awareness of proper posture, decreased activation and strength of the core muscles, increased time spent sitting, and sedentary lifestyles. The insufficient use of the core abdominal muscles, in particular the transverse abdominis, and lack of body mechanic awareness during activity, is associated with increased predisposition for injury including low back pain, the development of hernias in adults and protrusion of the abdomen. Weakness may occur for various reasons including abdominal stretching, such as occurs with weight gain and pregnancy, and with incorrect technique with or incorrect selection of abdominal exercises.

Typical exercises alone may not be enough to improve core muscle awareness and activation. The difference between the amount of time spent exercising compared to the amount of time spent without active tensing of the core is significant. Learned, automatic actions are a product of subcortical structures. In order to improve automaticity and habit formation of a behavior (e.g., subconscious contraction of the core), significant attention and effort is required, which depends on the cortex of the brain. The more something is practiced, the more it is learned. Such repetition that is required for retraining of the core abdominal muscles, in particular the transverse abdominis, in corrected postures is not typically achieved through typical daily activity or exercise, thus resulting in ineffective retraining and a need for this product. The ability to target, stimulate and contract one or more muscles in appropriate postures repeatedly throughout the day with everyday activities will better improve user awareness and activation of the intended region (e.g., core abdominal muscles with emphasis on the transverse abdominis, etc.) and ultimately improve subconscious activation by the user.

There have been various methods and devices for improving posture and for activating and strengthening the abdominal muscles. However compared to other devices, various embodiments discussed herein can facilitate training one or multiple intended region(s), e.g., the core abdominal muscles with emphasis on the transverse abdominis, in appropriate postures in response to the device providing a stimulation via one or a plurality of modules with one or a plurality of alerting mechanisms that can give a stimulation in a segmental and/or a simultaneous manner, to a segment(s) or the span of the intended one or multiple regions of the body, and said modules of one or more regions can operate together or separately.

### SUMMARY OF THE INVENTION

The following presents a simplified summary in order to provide a basic understanding of some example aspects of the disclosure. This summary is not an extensive overview. Moreover, this summary is not intended to identify critical elements of the disclosure nor delineate the scope of the disclosure. The sole purpose of the summary is to present some concepts in simplified form as a prelude to the more detailed description that is presented later.

According to one aspect, an example device is disclosed in which to facilitate the awareness and the activation of the (core abdominal) muscles in a corrected posture. The example device comprises: one or more modules, wherein each module of the one or more modules is configured to be positioned adjacent to one or more associated muscles or regions of a user; one or more alerting mechanisms configured to generate an alert to the user wherein a first alerting mechanism of the one or more alerting mechanisms comprises an electrically powered device producing one or more of a vibration, a visual output, an audible sound, a sensory level electrical stimulation, and a notification from a personal computer and/or smart device (e.g., smart phone, smart watch, portable device/tablet); one or more attachment element(s) configured to attach the device to the user; a controller configured to operate the device.

According to another aspect, a method is disclosed for facilitating the awareness and activation of one or more muscles in appropriate postures, comprising: alerting a user to a correct posture via one or more of a computer or a smart device; facilitating user activation of muscles of one or more regions of a body of the user via one or more alerts generated via the one or more of the computer or the smart device, wherein the one or more alerts facilitate one of sequential or simultaneous user activation of the muscles in a correct posture; and the one or more alerts facilitate one of sequential or simultaneous user activation of the muscles in a correct posture via one or more of a computer and a smart device (smart phone, smart watch, portable device, e.g., tablet); and providing instruction to the user to correct posture and activate one or more of at least one region of the body or the one or more muscles via one or more of a computer or a smart device.

To accomplish the foregoing and related ends, certain illustrative aspects of the disclosure are described herein in connection with the following description and the drawings. These aspects are indicative, however, of but a few of the various ways in which the principles of the disclosure can be employed and the subject disclosure is intended to include all such aspects and their equivalents. Other advantages and features of the disclosure will become apparent from the following detailed description of the disclosure when considered in conjunction with the drawings.

### BRIEF DESCRIPTION OF DRAWINGS

Objectives, features and advantages of various embodiments will be illustrated by the following drawings. It is to be understood that the following drawings serve as examples and do not limit the scope of the invention. While specific embodiments of the disclosure are described herein, it should be understood that the disclosed device, systems, methods and apparatuses are not so limited and modifications may be made without departing from the scope of the present disclosure. The scope of the device, systems, methods, and apparatuses are herein described, referred to, exemplified or shown, and all features, devices, processes, and methods that come within the meaning, either literally or by equivalence, are intended to be embraced therein.

FIG. 1a shows an embodiment of the modules on an anatomical image of the trunk relative to the transverse abdominis muscle.

FIG. 1b shows schematic drawings of various embodiments of the modules with one or multiple alerting mechanisms (e.g., vibratory motors) per module in varying arrangement, in which the general placement of the modules on the trunk would be unchanged.

FIG. 1c is a schematic drawing representing an embodiment of wireless and/or wired controllers.

FIG. 2a shows an embodiment of the modules on the abdomen of a user in actual physical likeness.

FIG. 2b illustrates an embodiment of an accessory garment to further secure the modules to a user in actual physical likeness.

FIG. 3 is a schematic drawing of an embodiment of the modules on an anatomical image of the trunk illustrating a segmental (e.g., ascending/descending) effect pattern of activation.

FIG. 4 illustrates an embodiment of the modules placed on the abdomen of a user in actual physical likeness with use of a smartphone as the controller, displaying the syncing of the modules on the screen.

FIGS. 5a-d are schematic drawings representing embodiments of wired and/or wireless controllers (also FIG. 1c) and may comprise a smartphone (FIG. 5a, also FIGS. 1c, 4), a smart tablet (FIG. 5b) and/or a smart watch (FIG. 5c) which can download an associated application software (“app”) to send and receive communication to and from the modules via wireless signal or network connection. FIG. 5d shows embodiments of wired and/or wireless controllers (also FIG. 1c) designed specifically for invention operation in connection with various embodiments.

FIG. 6a is a perspective view of an embodiment of the exterior of the module with a housing element.

FIG. 6b is an exploded view of an embodiment of the module with a housing element.

FIG. 6c illustrates the use of a smartphone as the controller with an embodiment of adjustable parameters of the stimulation (e.g., vibratory) on the screen.

FIG. 7a is a schematic drawing of an interconnecting element between the modules.

FIG. 7b illustrates an embodiment of the modules with the interconnecting element placed on the abdomen in human likeness.

FIGS. 8a-d illustrate an embodiment of additional modules placed on one or more posturally appropriate areas. FIGS. 8c-d illustrate poor and optimal standing and sitting posture, without and with modules, respectively.

FIGS. 9a-d illustrate embodiments of an accessory garment to further secure the modules to the user. FIG. 9a shows an embodiment of a “tube” like garment. FIG. 9b shows an embodiment of a wrap type garment, in which one end would secure to the other end. FIG. 9c shows an embodiment of a wrap type garment taking the shape as when it would be secured from one end to the other. FIG. 9d shows an interior view of a garment with an embodiment for pockets or slots for the modules, in addition to vertical stays to help maintain shape and prevent buckling and use of specific material at the superior and inferior aspects to prevent sliding.

FIGS. 10a-c show an embodiment of flow between the user, controller and modules for an example method, according to various aspects discussed herein.

FIGS. 11a-c show an embodiment of flow of an example method with use of additional modules located in posturally indicated areas used during the preparatory phase (i.e., preparatory posture correction phase) preceding the training phase (i.e., abdominal training phase).

FIG. 12a,b show an embodiment of flow of an example method for instruction and performance via training on a smart device/two paired smart devices, i.e., a smartphone and a smartwatch without use of the modules.

It should be noted that the drawings are diagrammatic and not drawn to scale. Relative dimensions and proportions of parts of the figures have been shown exaggerated or reduced in size for the sake of clarity and convenience in the drawings. The same reference numbers are generally used to refer to corresponding or similar features in the different embodiments, except where clear from context that same reference numbers refer to disparate features. Accordingly, the drawings and description are to be regarded as illustrative in nature and not as restrictive.

While embodiments of the disclosure pertaining to providing system(s), method(s), etc. for encouraging activation and/or awareness of muscles are described herein, it should be understood that the disclosed machines, electronic and computing devices and methods are not so limited and modifications may be made without departing from the scope of the present disclosure. The scope of the systems, methods, and electronic and computing devices for encouraging activation and/or awareness of muscles are defined by the appended claims, and all devices, processes, and methods that come within the meaning of the claims, either literally or by equivalence, are intended to be embraced therein.

### DETAILED DESCRIPTION OF THE INVENTION

Example embodiments that incorporate one or more aspects of the present disclosure are described and illustrated in the drawings. These illustrated examples are not intended to be a limitation on the present disclosure. For example, one or more aspects of the present disclosure can be utilized in other embodiments and even other types of devices. Moreover, certain terminology is used herein for convenience only and is not to be taken as a limitation on the present disclosure. Still further, in the drawings, the same reference numerals are employed for designating the same elements.

This disclosure relates to a wearable device designed to improve the activation and awareness of at least one region of the body (e.g., the core abdominal muscles with emphasis on the transverse abdominis and/or one or more other regions of the body) in appropriate postures. In particular, this disclosure relates to a device worn by the user that provides a means to alert the user to activate at least one region of the body (e.g., to activate the core abdominal muscles with emphasis on the transverse abdominis in appropriate postures). Various embodiments can accomplish this through stimulation (i.e., vibratory/haptic, sensory level NMES, etc.) applied to a segment(s) or the span of the appropriate region, for example the abdominal region, which prompts the user to activate the muscle, for example the transverse abdominis, in response to the stimulation. Embodiments also include an instructional training method including how to correctly engage the muscle (s), for example the core abdominal muscles with emphasis on the transverse abdominis, in appropriate postures.

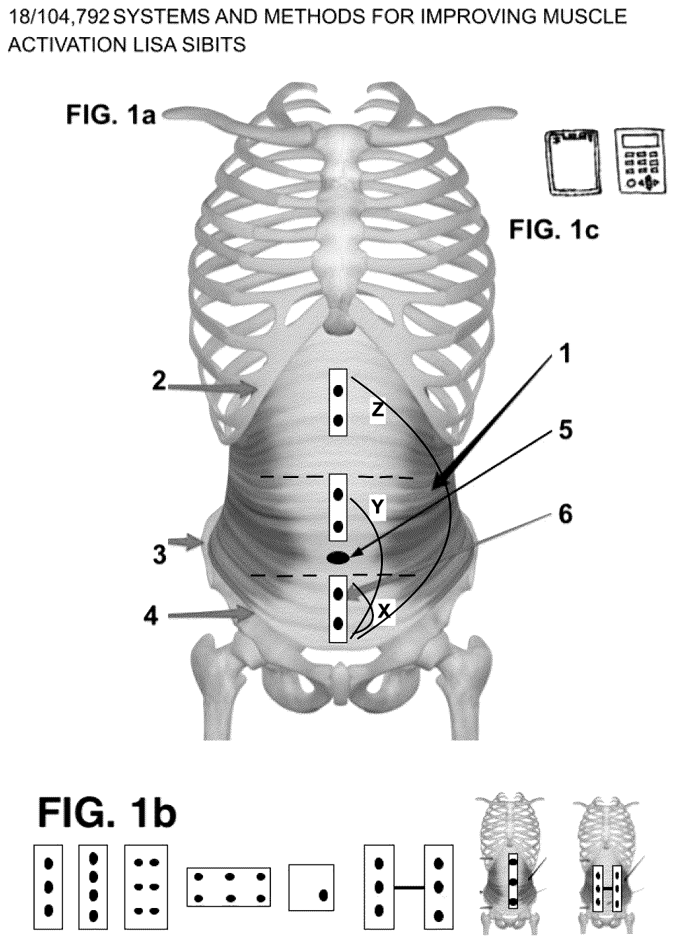

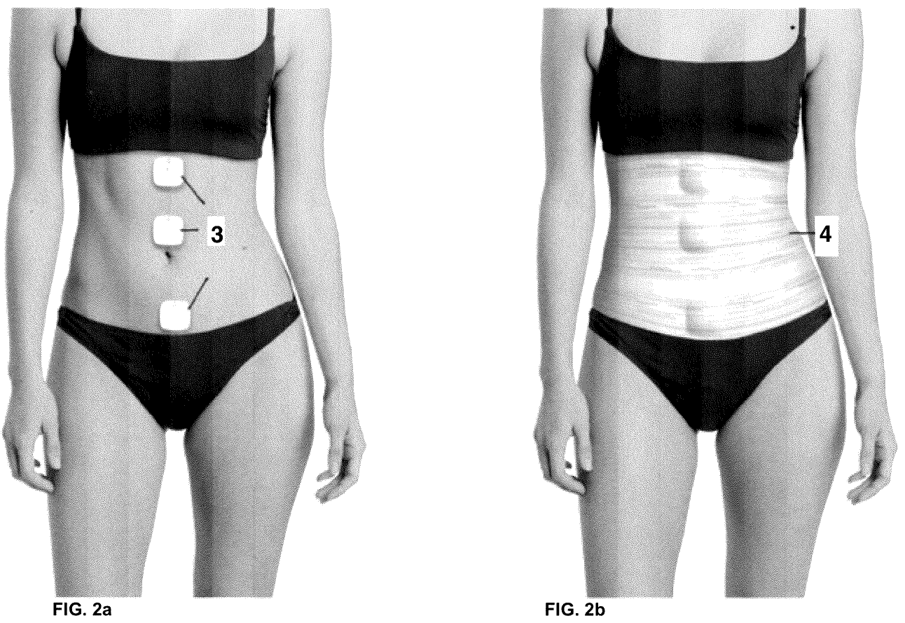

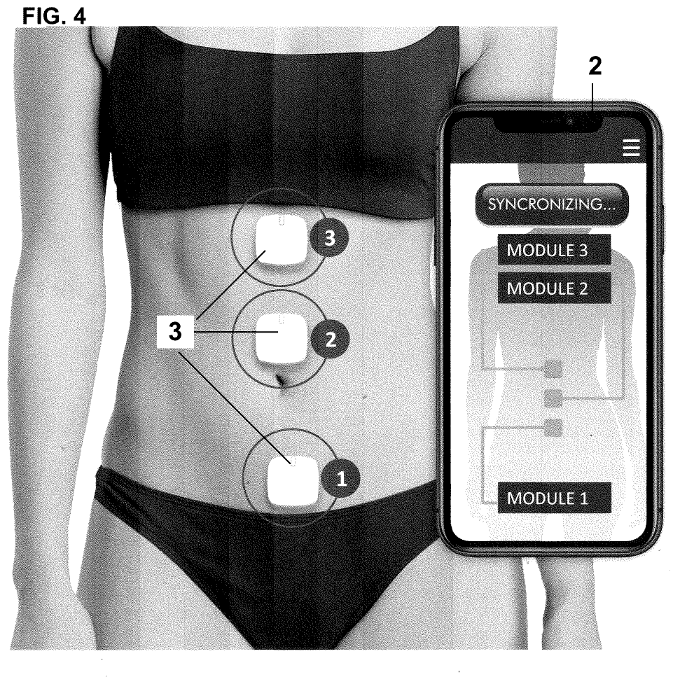

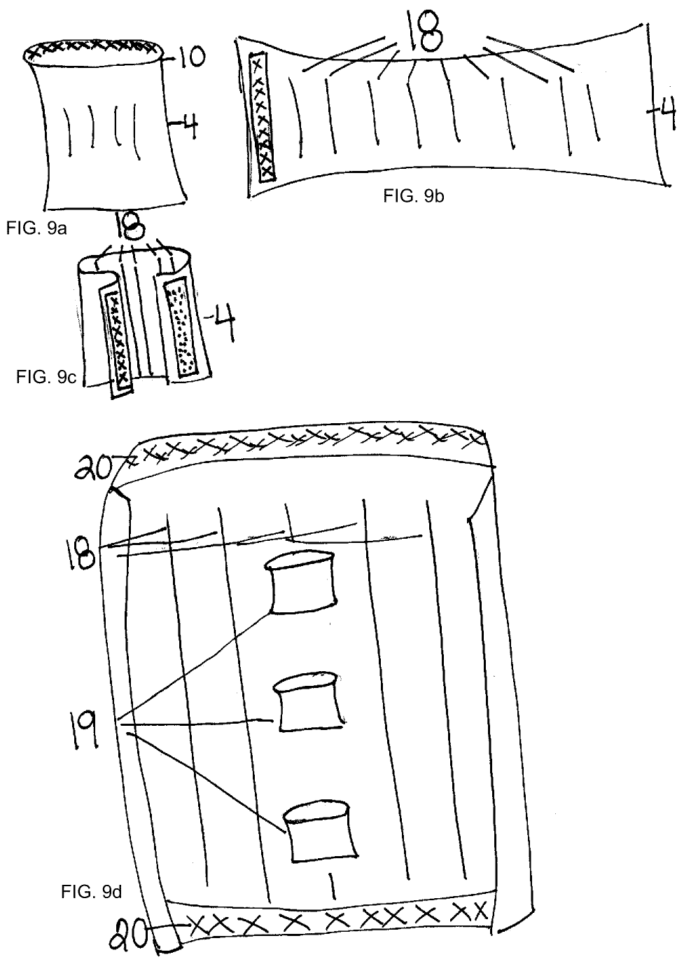

Referring to FIGS. 1, 4, 6 illustrated is a first example embodiment, comprising a controller 2 (also FIGS. 1c, 4, 5a-5d, 6c) and one or more modules 3 (also FIGS. 1a, 1b, 2a, 3, 4, 6a, 6b, 7a, 7b, 8c, 8d) which are also referred to as transmitter modules 3 interchangeably. The transmitter unit(s) 3 can be placed on or adjacent to muscles for which activation is to be trained. While various embodiments can relate to any of a variety of muscles and/or muscle groups, for the purposes of illustration, various examples discussed herein are described in connection with training activation of core abdominal muscles with emphasis on the transverse abdominis. Referring to FIG. 1., the transverse abdominis muscle is indicated as 1. The origins of the transverse abdominis muscles are the costal cartilages of ribs 7-12 (indicated as 2), the thoracolumbar aponeurosis (not shown), the iliac crest (indicated as 3) and the inguinal ligament (indicated as 4). The insertions of the transverse abdominis are the abdominal aponeurosis (indicated as 6; the arrow points underneath the module) and linea alba (not shown). The umbilicus is indicated as 5. Thus, in various such example embodiments, the module(s) 3 can be placed on the abdomen overlying the location of the TA muscle (FIGS. 1a, 1b, 2a, 3, 4, 7b, 8a, 8c, 8d). The device may also include an accessory garment 4 of varying embodiments (see, e.g., FIGS. 2b, 9) to further secure the modules 3 to the user. Various embodiments can also include a training method which may occur via instruction of any applicable method including one or more of, print, audio, video, web or app based and/or audio-visual based format.

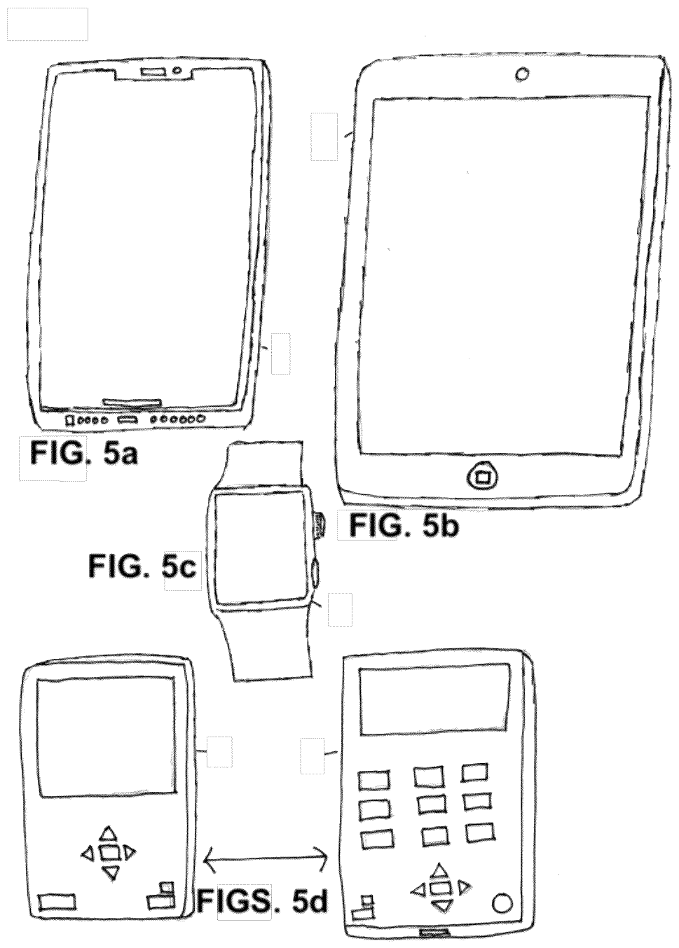

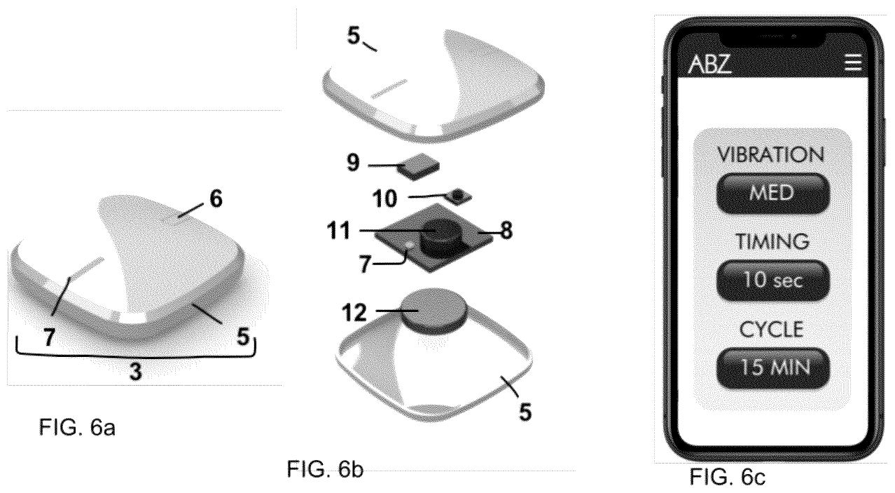

The controller 2 is configured to direct the operation of the device. However, the device may also be manufactured so that the modules can be operated without an external controller. The controller 2 may be any of the following: a wired/wireless device manufactured specifically for operation of various embodiments (FIGS. 1c, 5d), and/or a smart device/smart devices, e.g. smart phone/smart tablet/smart watch) (FIGS. 1c, 4, 5a-c, 6c) that are paired to operate together and are equipped with the appropriate operating software/app. Said wired/wireless device manufactured specifically for operation of various embodiments may be comprised of, but is not limited to: one or more of connection areas/sockets/ports, display, microprocessor/microcontroller, rechargeable battery, memory, wireless operation capacity, etc., as applicable. Either controller 2 option can have preset programs with the ability for the user to adjust certain parameters (FIGS. 6c, 10b, 11a-b) and/or a manual setup option. Additionally, the controller 2 may have the capacity to track and report various aspects of performance to the user (e.g., repetitions, duration, muscle activity, etc.) via any of a variety of methods (e.g., USB or wireless transmission). The controller software or app can have the ability to be updated via wired or wireless means and either controller option can have the ability to be charged.

Referring to FIG. 6a, illustrated is an exemplary embodiment of an external view of the module. The module(s) transmit the intended alert to the user. Said transmitted alert may be a vibration, a visual output, an audible sound, sensory level electrical stimulation and/or communication with a personal computer and/or a smart device. The modules 3 may include a housing element 5, with an on/off button 6 or switch, and one or more LED indicators 7. The housing elements 5 can be constructed from varying material as applicable (e.g., plastic, rubber, metal, ceramic, etc.). On the interior (FIG. 6b), additional components may include a printed circuit board (PCB) 8, wireless transceiver/connectivity 9 (e.g., employing any of a variety of wireless communication technologies, such as Bluetooth, etc.), a microtimer and/or microswitch 10, one or more haptic vibration motors 11, one or more LED indicators 7, a rechargeable battery 12, one or more microprocessors/microcontrollers (not shown) with appropriate means for memory, (e.g., such that the modules may operate without the controller) etc., and appropriate components for operation without controller as appropriate. There may be one or more input and/or output (I/O) components (not shown) as appropriate, such as to charge via wired means or there may be an inductive coil (not shown) or other means as applicable, to charge through electromagnetic induction. Also not shown and which may be included are one or more of a haptic driver, a battery charge controller and other unspecified components, as appropriate. The driver may be an external differential amplifier or integrated into a single IC along with a haptics processor. In other embodiments, the one or more vibration motors 11 may be replaced with necessary components for other types of alerts (e.g., visual, auditory, NMES, etc.). In an embodiment, the modules may include biofeedback capacity, (e.g., EMG or FSR to detect muscle contraction; accelerometer to detect linear motion; gyroscope sensor to detect movement and position, etc.) and the ability to send the information to the controller.

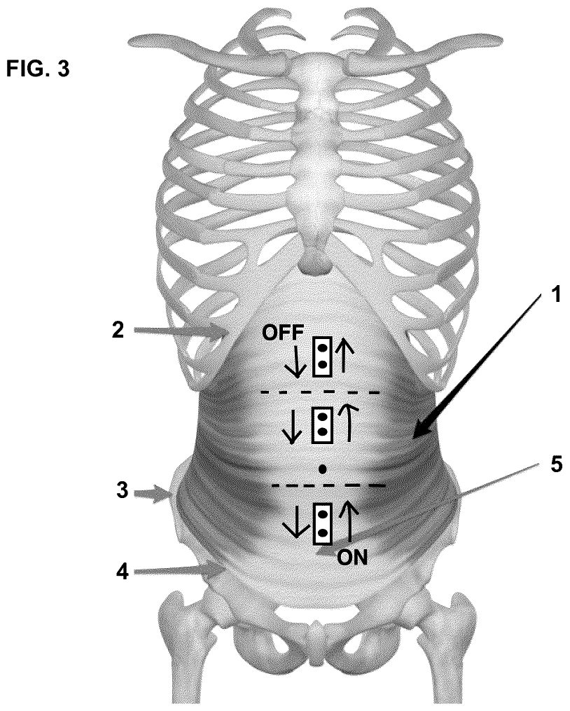

Embodiments of the modules 3 are illustrated in FIGS. 1a, 1b, 2, 3, 4, 6b, 7a. FIG. 1b shows schematic drawings of various embodiments of the modules 3 with one or multiple haptic transmitter motors 11 in varying arrangement (see also FIG. 1a). The number of modules 3 and the number of motors 11 per module may vary as indicated to achieve the desired stimulation effect. The last drawing of FIG. 1b shows an example of the modules 3 positioned in pairs on the abdomen in human likeness; the pairs are shown oriented vertically and parallel to one another, one of each pair just to the outside of midline bilaterally, with the pairs extending vertically above and below the umbilicus. Pertaining to usage for core abdominal training, the module(s) 3 can be positioned at the midline of the abdomen in a vertical fashion above and below the umbilicus (FIGS. 1a, 1b, 2a, 3, 4, 7b, 8a, 8c, 8d). Referring to FIG. 1a, the user may vary the number of modules 3 as preferred; in this particular example for core abdominal retraining, the user may choose to use one module (indicated as “x”) to target the inferior most region, or two modules (indicated as “y”) to target the lower and mid region, or all three (indicated as “z”) modules 3 in order to target a particular region of the core abdominals. FIG. 3 is a schematic drawing of an embodiment of the modules 3 illustrating the segmental “zipper” effect pattern of activation. In an embodiment, the user could choose for the modules to be activated simultaneously.

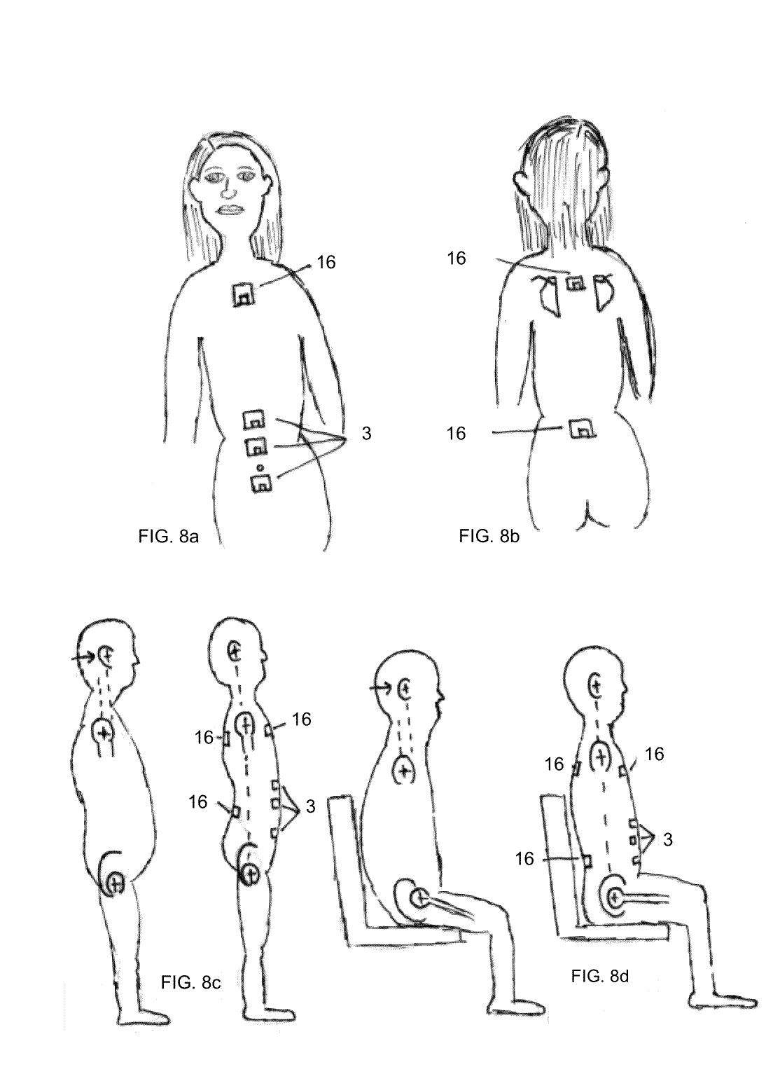

In an embodiment, there may also be additional modules 16 that may be placed on one or more appropriate (e.g., posturally appropriate, etc.) areas (see FIG. 8) such as, but not limited to, the sternum, interscapular region and/or low back. The additional modules 16 may be comprised of any variation of indicated components as the modules 3. The additional modules 16 may be used to enhance the user's positioning (e.g., posture, etc.) during the preparatory phase (e.g., preparatory posture correction phase, etc.) prior to beginning the muscle (e.g., abdominal, etc.) activation (e.g., training phase). In another embodiment the additional modules 16 (e.g., posture modules) may be selectively chosen to be used at varying times independently from the training modules 3, such as if the user chooses to use the additional modules 16 to receive additional reminders to improve awareness (e.g., posture awareness) between the training (e.g., core abdominal training, etc.) sessions.

The additional modules 16 may also be placed in other areas as appropriate to facilitate positioning with use. For example, a Physical or Occupational Therapist may observe frequent lateral leaning to the right of the thoracic or lumbar region, or scapular protraction at rest and with reaching, and may recommend corresponding placement of an additional preparatory module(s) in order to increase the user's awareness and correction during the preparatory phase or between the training phase.

The modules and/or additional posture/positioning modules may be used in limitless other regions as appropriate. For example, a Physical or Occupational Therapist may observe scapular protraction and thoracic flexion at rest and/or with upper extremity reaching and may recommend for preparatory use, placement of the additional modules 16 on the interscapular region and/or sternum for preparatory position correction and the use of training modules 3 placed on the upper arm or deltoid for the training phase (i.e., reaching in this example).

There exists multiple options to secure the modules 3 against the skin of the user in singularity or in plurality. In one embodiment, the modules 3 may be secured with a removable, reusable, replaceable securing mechanism. Said securing mechanism may have two faces. In an embodiment, said securing mechanism may have two faces, which can comprise adhesive on both sides. The modules 3 may be secured against the skin of the user with one face of the adhesive while said second face is secured against the module. In an alternative embodiment and/or additional usage, said securing mechanism may be an additional type of securing mechanism with two faces, in which one face may be comprised of adhesive and a second face consisting of a first component of a hook and loop fastener, a clip, snap, or a magnet, etc. Said securing mechanism with one face comprised of adhesive, first component of a hook and loop fastener, a first arm of a clip, a first component of a snap or a magnet, etc., may attach to said module via a second face of adhesive, a second component of a hook and loop fastener, a second arm of a clip, a second component of a snap or a magnet, etc.

In another embodiment, the modules 3 may be secured with in addition to and/or separately from a snug fitting wearable garment 4 or wrap (see e.g., FIGS. 2b, 9) of limitless embodiments. Said garment 4 may be an undergarment similar to a tank top that is intended to fit snugly against the skin and may be composed of materials chosen for varying degrees of compression and extensibility as desired. Furthermore, said garment 4 may be a “tube” type unit (see e.g., FIG. 9a) or may be a wrap/belt or band (see e.g., FIGS. 9b, 9c) with appropriate means to connect one end to the other to secure the wrap/belt or band around the relevant region (e.g., the torso) of the user (i.e., velcro, clasps, snaps, magnets, etc.).

The modules 3 may have the ability to be attached to and removed from said snug fitting wearable garment 4 through various means, including but not limited to, said second component of hook and loop fastener, Said hooks, clips, snaps, magnets etc., may be woven into or attached to a wearable garment 4 via a second face of adhesive, a second component of clip, magnet, etc.

Said garment 4 may include areas composed of specific material 20 to prevent sliding (see e.g., FIG. 9a, 9d), and/or vertical stays 18 in various positions (see FIG. 9) to prevent buckling and to maintain adequate level of rigidity. The garment 4 may further secure the modules 3 due to the snug fitting nature and composition of the material and/or due to additional design elements, such as a slot or pocket 19 within the garment 4 (FIG. 9d) or through the use of attachment elements as stated above.

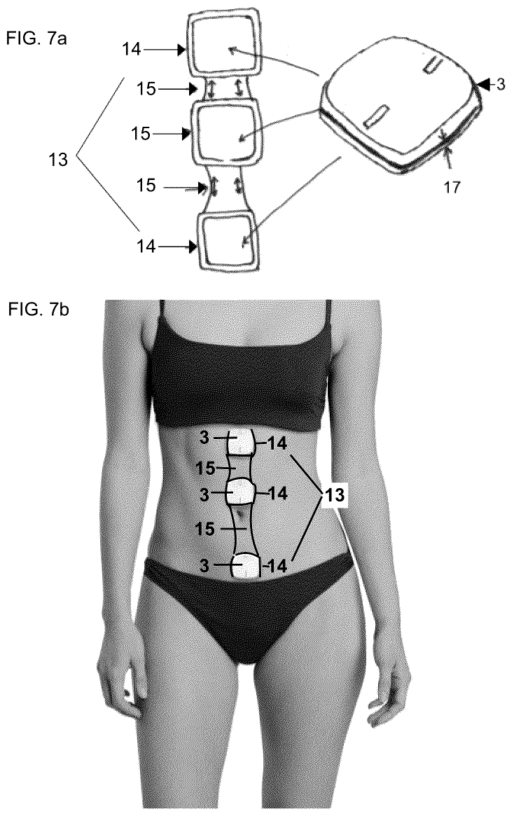

Referring to FIG. 7a, in an embodiment, the multiple modules 3 may be secured (snapped, slid etc.) into a module interconnecting unit 13 with appropriately shaped and number of ring-like openings 14 specifically manufactured to hold the intended number of said modules 3. The module interconnecting unit 13 may be designed to press, stretch around, slide or snap etc., the modules 3 in order to securely connect multiple modules 3. The housing element 5 of the modules 3 may have groove(s) 17 along the outside of the housing 5 in which said ring-like elements 14 of module interconnecting unit 13 would fit into. Said ring-like openings 14 of said module interconnecting elements 13 may have the ability to be detached from one another in the instance the user desires to vary the number of modules 3 used. Said ring-like elements 14 may be made from a liquid plastic, rubber or any other material deemed appropriate, chosen for extensibility and texture as desired. Between said ring-like openings 14 may be a material 15 that connects said ring-like openings 14 to one another. Said material may be made from a liquid plastic, elastic or non-elastic material, or from any other suitable material, chosen for extensibility and texture as desired. Referring to FIG. 7b, use of said module interconnecting unit 13 would allow the user to place the desired number of the modules 3 on the intended area (in this example, the abdomen) as one rather than placing each individually and serves as another means of securing the modules 3.

### Operation

The controller 2 may send commands to and receive communication from, through wireless, i.e. bluetooth, network etc, and/or wired means, the microcontroller/processor(s) of the modules 3, 16. In the embodiment for stimulatory output to be a vibration, a mechanism such as a timer or microswitch (not shown) can connect the battery 12 to the motor 11 and signals the microcontroller/processor which signals the haptic driver to drive the actuator which delivers the vibration output to the user.

There can be preparatory stimulation provided prior to the training session, such as to alert the user to correct positioning or posture prior to the imminent training session. The preparatory phase (e.g., preparatory posture correction phase, etc.) can have a specific corresponding stimulation alert and/or associated parameters including duration, some of which may also be adjustable in an embodiment. In an embodiment, there may also be additional modules 16 comprised of the components of the modules 3.

During the training phase, the synchronized modules 3 (e.g., abdominal modules) may be activated in a segmental manner (e.g., ascending) or all at once as designated by the preset program or user adjusted parameters. The stimulation (e.g., vibration or sensory level NMES) will last for the designated duration. The stimulation (e.g., vibration or sensory level NMES) can stop in a segmental manner (e.g., descending) or in an option all at once. There is a predesignated amount of time between repetitions, or in an embodiment, modifiable by the user. The training intervals continue to repeat until length of time or number of repetitions, as per preset program or in an embodiment as modified by the user selected/indicated, is complete. Referring to FIG. 3, the segmental method of activation is shown. The modules may turn on one module at a time in an ascending manner, stay on for the designated duration and then turn off one module at a time in a descending manner. FIG. 3 also illustrates the transverse abdominis muscle, its origins and insertions.

In an embodiment, the modules 3 may include biofeedback capacity, such as electromyography (EMG) or FSR to detect muscle contraction, and the ability to send the information to the controller. The controller 2 may have the capacity to report various aspects of performance summary to the user (e.g., repetitions, duration etc.).

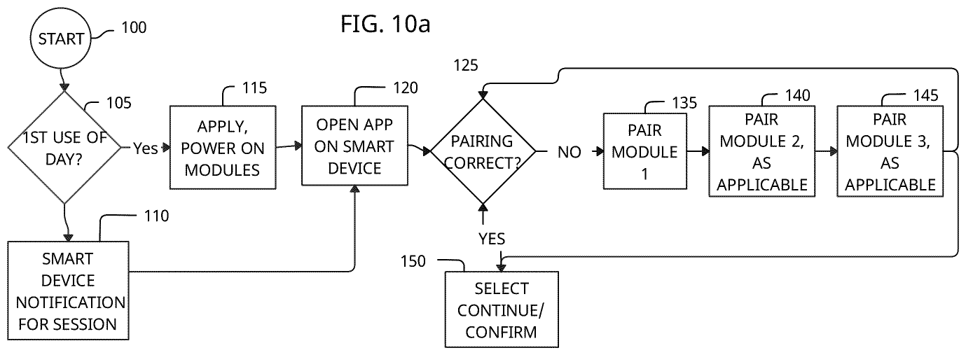

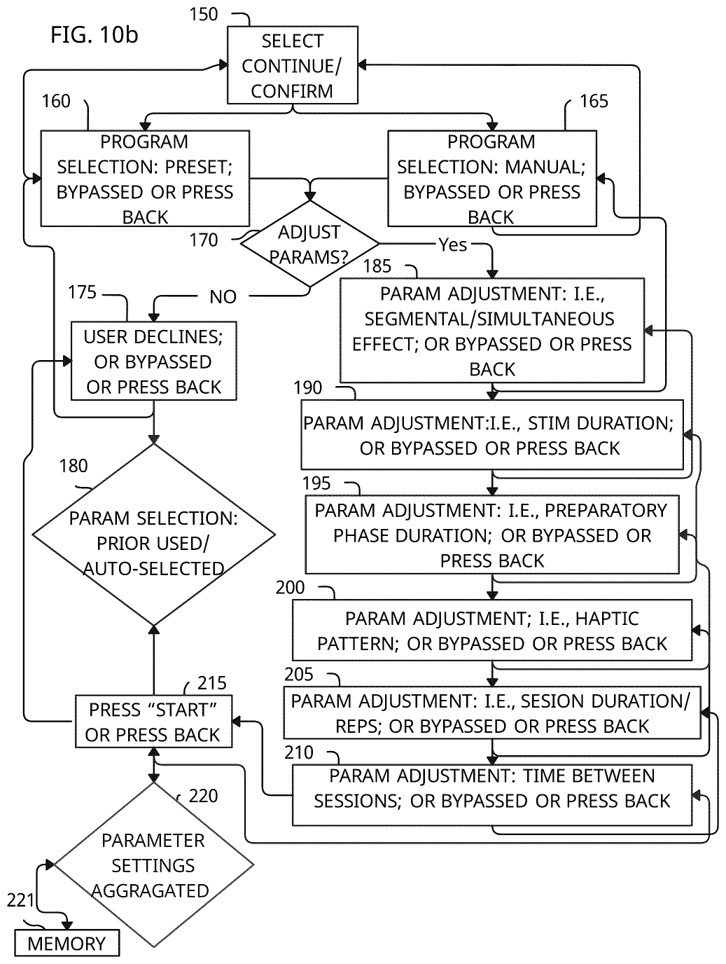

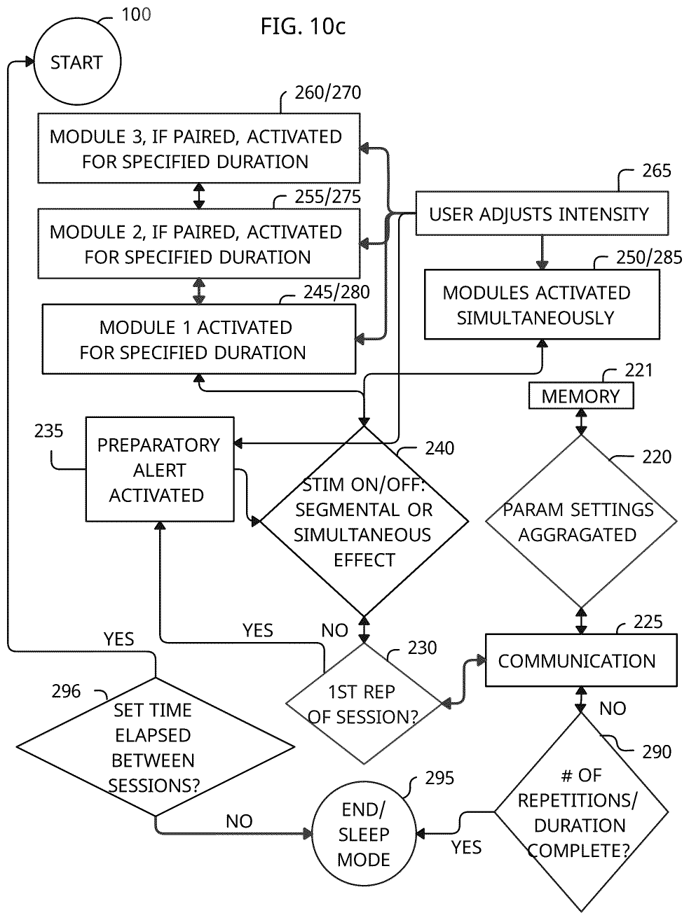

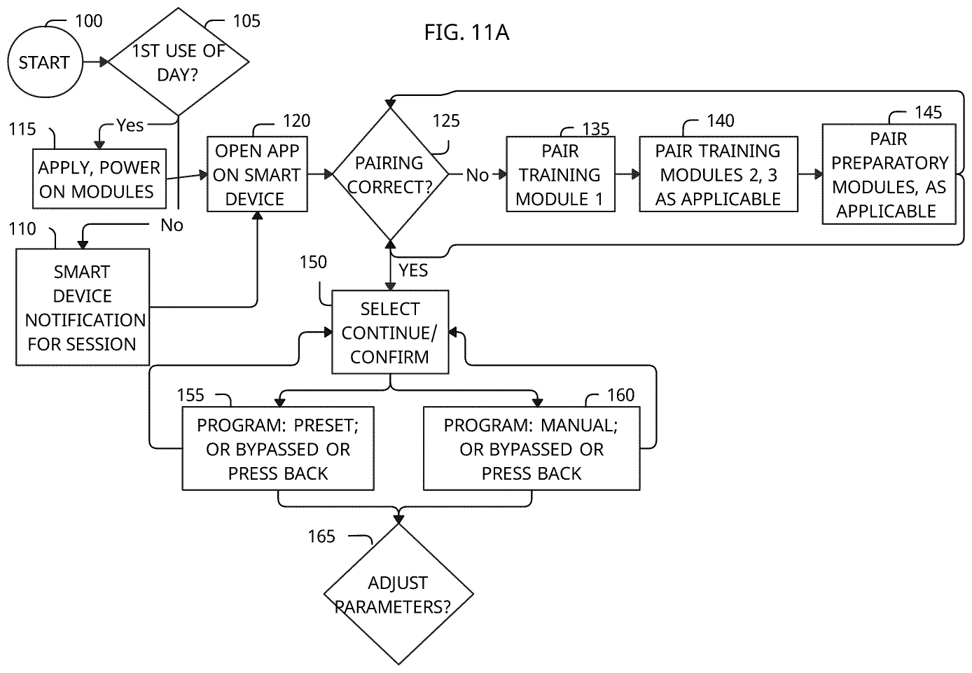

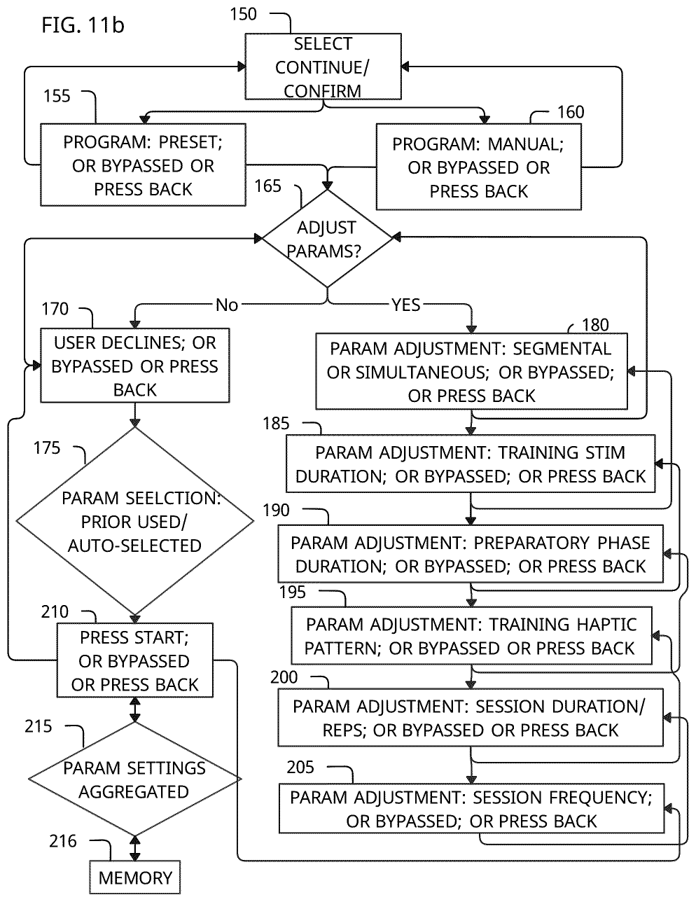

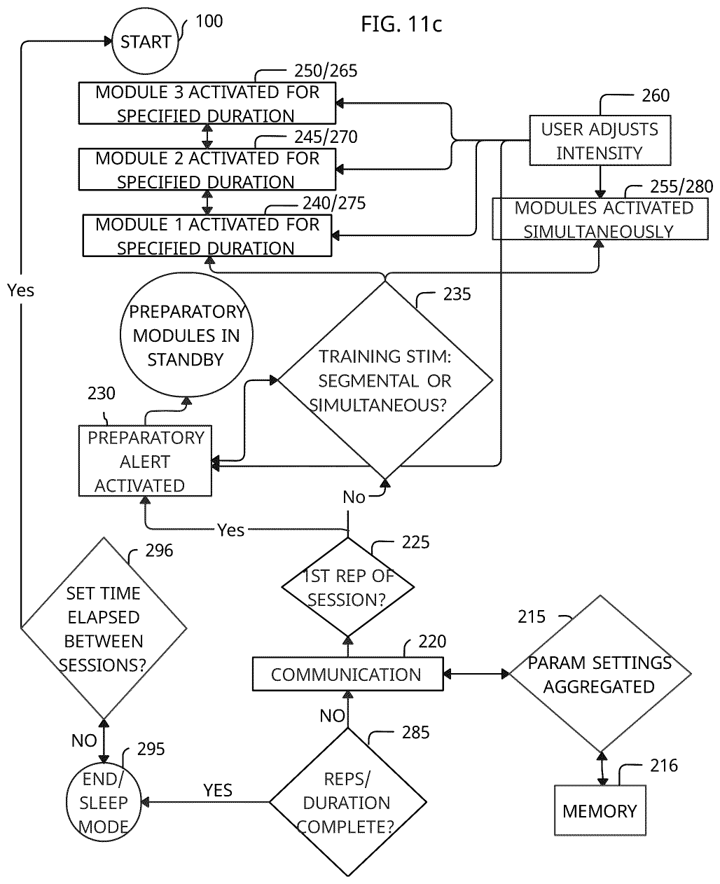

In additional embodiments, there may exist any relevant components such as but not limited to, memory etc., on the modules/modules 3, 16 to enable the ability to be operated independently from the external controller 2 (see 221, FIGS. 10b-c, see 216 FIG. 11b,c).

The system may include multiple preset programs and/or a manual mode. The stimulation parameters of the associated stimulation may vary based upon pre-set programs or parameters adjustable by the user in order to decrease desensitization/habituation. The parameters can include but are not limited to one or more of: haptic type/pattern “character” (when said stimulation is a vibration), intensity, frequency, ramp-on and ramp-off time (with segmental effect: time for stimulation of each segment and time for entire region; on in segmental fashion vs all on at same time; off in segmental fashion vs all off at same time), on-time duration (length of time stimulation is on), off-time duration (time between stimulation repetitions), session duration (length of session or number of reps), preparatory phase duration (e.g., preparatory posture correction duration, etc.), etc. See FIG. 10 for an exemplary flowchart.

The number of modules 3 can be chosen by the user and correspond to the intended muscle (e.g., segments of the abdominals/TA, etc.) (see FIG. 1a). If the user intends to target the entire area of the abdominals/TA three modules may be applied (indicated as Z), however, if just the lower or lower and middle segments are desired, then the corresponding module(s) are applied accordingly, indicated as X and Y respectively. In various embodiments, for the first use of the day, the modules 3 can be attached to the user as appropriate, in one embodiment via reusable adhesive, and left in place for the duration of the day and obscured from view by normal clothing, better obscured by loose fitting garments rather than snug fitting. The modules 3 may be further secured with an accessory garment/wrap 4 (FIG. 2b, 9) and/or module interconnecting unit 13 (FIG. 7).

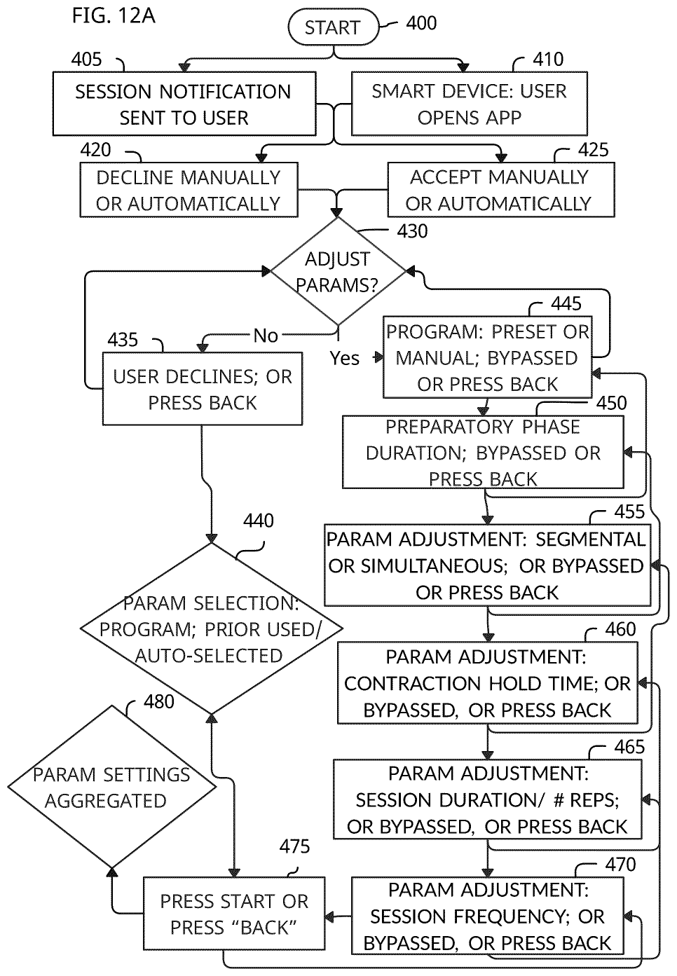

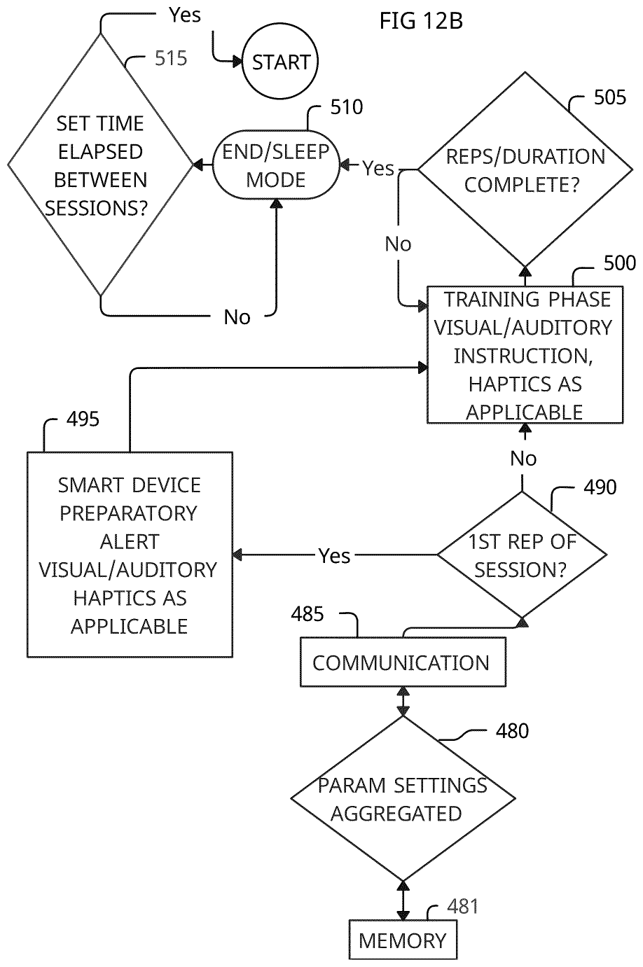

In an embodiment, the awareness and activation of one or more of a region of the body and an intended muscle (e.g., core abdominal muscles with emphasis on the transverse abdominis) in a corrected posture is facilitated by one or more of a computer or smart device (smart phone, smart watch, portable device, e.g., tablet) without use of modules. See FIGS. 12a-b for an exemplary flowchart. One or more of a computer or smart device (provides an alert and instruction (via at least one of auditory, visual, interactive training, use of an app, etc.). An alert is provided of the imminent training session, in which the user can accept or decline, or in an embodiment is automatically selected or declined if no response is provided. Prior to the training session taking place, an alert is provided for a preparatory posture or position correction phase in which the user corrects posture or position prior to the training of the intended muscle (e.g., core abdominal muscles with emphasis on the transverse abdominis). There is a predetermined amount of time in which to correct posture, which in an embodiment is modifiable. After the preparatory (e.g., posture correction) phase concludes, the training phase begins. During the training phase, the intended muscle (e.g., core abdominal muscles with emphasis on the transverse abdominis) may be activated in a segmental manner (e.g., ascending) or all at once as designated by the preset program or user adjusted parameters. The activation will last for the designated duration and can be released in a segmental manner (e.g., descending) or in an option all at once. There is a predesignated amount of time between repetitions, or in an embodiment, modifiable by the user. The training intervals continue to repeat until the duration or number of repetitions, as per preset program or in an embodiment as modified by the user, is complete. Upon completion of the training session the app goes into standby until the next predetermined session, which in an embodiment may be modified by the user. At the start of the next session, the entire cycle repeats.

FIGS. 10a-c show one example embodiment of a method of operation of the device between the controller 2, in which said controller is a smart device, and the modules 3 (e.g., core abdominal modules, etc.) (refer to FIGS. 1a-c, 4, 6 for specified components). The corresponding app can be opened on the smart device 2, the modules 3 can be turned on 115 and the app can be opened 120; the command to begin pairing 125 can be selected on the app, versus automatically recognized 125 when pairing is indicated, in an embodiment. The modules 3 can be paired by pressing and holding the button or recognized from prior use. The process can start with one module 135 (in the embodiment for use for core abdominal training, the most inferior module) and can be continued in a segmental order 140, 145 (in the embodiment for use for core abdominal training, in an ascending order) for the desired number of modules (alternatively, a descending order starting from an upper). The pairing in segmental order may be significant for operation using the segmental effect of activation, although may also be used in simultaneous mode with this pairing. The number of modules 3 can be confirmed 150 or corrected 150. If an error occurs the user can return back to the pairing screen 125 and continue the pairing process.

After pairing is completed vs recognized, the user can be prompted to adjust or decline adjustment of various parameters, beginning with, by means of example, selecting the option for a preset program or manual mode 160, 165 or to bypass in which case the program will be auto-selected, for example at random vs last used. After declining or selecting the option for a preset program or manual mode 160, 165, the user can have the option to modify various parameters 170 through 210 or to bypass and use last selected/prior used vs auto-selected/random selection and may designate an option as default. As progressed through parameter adjustment, the user may have the ability at any time to choose “back” to return to the previous selection. The various modifiable parameters may include, for example and in no specific order, amount of time for the preparatory phase (e.g., preparatory posture correction phase, etc.), which may include the option for the adjustment of a corresponding stim alert (e.g., haptic) and associated parameters, some of which may also be adjustable in an embodiment; stimulation on/off pattern (segmental (e.g., ascending/descending) or simultaneous); session duration: number of repetitions or time duration—i.e., 10 reps vs 10 minutes; session occurrence/frequency (i.e., every 15/30/60 min, etc.); stimulation duration/′ on time” (once all activated, i.e., when segmental pattern is selected) i.e., 10/15/30/60 sec, etc.; training phase haptic pattern/character i.e.; a particular pattern of haptic stimulation; etc., in the instance when said stimulation is a vibration or TENS/sensory level NMES. Alternatively, the modules 3 have the ability to be operated and parameters selected independently from the controller.

Once parameter adjustment is completed, the user can select “start” 215. The parameter settings can be aggregated at the level of the controller or the modules 220. Communication/direction 225 can then be sent wirelessly between the controller/smart device 2, [and] [to] the modules 3 as applicable, and memory 221. A preparatory alert stimulation 235 ((i.e., vibratory sequence or TENS/sensory level NMES sequence) (e.g., a “preparatory posture correction alert”, etc.) can be sent from the modules 3 and/or 16 and the user can have a predesignated amount of time in which the preparatory stimulation is occurring to adjust positioning (e.g., posture) in preparation for the imminent training session. The preparatory stimulation 235 can turn off after the designated time. The additional synchronized preparatory modules 16 may then go into standby mode. The training stimulation can then begin. The modules can vibrate either in a one-at-a-time segmental order, such as an ascending fashion 245, 255, 260, or simultaneously 250 according to the prior chosen option 240. The user may adjust the intensity of the stimulation at any time during the preparatory or training stimulation 265. The modules can continue to vibrate for the designated duration before turning off in either a segmental order such as a descending order 270, 275, 280, or simultaneously 285 according to the prior chosen option 240. Upon completion of the repetition 280, 285, communication can be sent back to the microcomputer/microprocessor determining if the training session is complete according to setup/parameters 290. If determined to be complete 295, the session ends. If determined not complete, the training sequence starts again 240 after predetermined “off time” (time between repetitions), and continues to repeat until length of time or number of repetitions, as selected/indicated in setup, is complete. The module(s) 3 can go into standby mode upon completion of the session 295 until the next session according to setup/parameters 296, or as desired by the user. At the start of the next session, the entire cycle repeats 100.

FIGS. 11a-c show a flow chart for an example method embodiment employing additional modules 16 placed at positionally (e.g., posturally, etc.) indicated locations, for example the sternum, interscapular and the lumbo-pelvic region, such as to provide additional tactile cues to improve performance and awareness of positioning (e.g., posture, etc.), (refer also to FIGS. 1a-c, 4, 6, 8 for specified components). The operation can be similar to that shown in connection with FIGS. 10a-c with the addition of the additional modules 16 (e.g., positioning/posture modules) which can be synced during the pairing process 145 and can vibrate during the preparatory phase 230 (e.g., preparatory posture stimulation phase, etc.). In an embodiment (flow not shown), the modules 3, 16 may be selected to be used separately such as if the user chooses to use the additional modules 16 to improve posture awareness between training (e.g., core abdominal training, etc.) sessions.

FIG. 12a,b show shows a flow chart for an example method embodiment without employing the modules 3, 16, with instruction and haptic vibration as applicable, provided by the smart device(s) 2 only (refer also to FIGS. 1a-c, 4, 6, 8 for specified components). The user can be provided instruction by the app. The instruction may include audio and/or visual training. Said smart device may also provide instruction haptic, visual and/or auditory feedback as indicated. In one instance, the user may perform via one smart device, such as a phone or a tablet with appropriate operating app installed. In another embodiment, the user may perform via a combination of smart devices, such as phone and a paired smart watch, again with appropriate operating app installed. In this instance, said paired smart device may provide audio/visual instruction and/or haptic feedback to alert the user of an imminent training session (i.e., preparatory posture correction phase, etc.) and of the training phase in which to perform and release the muscle (e.g., core abdominal, etc.) contraction. In either instance, whether use of one or use of more than one smart devices, the training session begins. The user can open the corresponding app on the smart device at will 410. In another embodiment, a training session notification can be sent to the user 405 at periodic or scheduled times/intervals or if the user has not performed in a certain amount of time. The user may decline or ignore the reminder 420 or accept 425 the training session. After the user opens the app or accepts the training notification, the user may then choose to adjust any parameters 430. If the user does not choose to adjust any parameters 435, the parameters can be dictated 440 as preset in the program or last used or randomly selected and the user presses start 475. If the user chooses to adjust parameters, the user can be taken through the applicable adjustable parameters 445 through 470 (i.e., preset or manual program, preparatory duration (e.g., preparatory posture correction duration, etc.); session duration: time vs number of reps; contraction duration/hold time; session frequency (i.e., 15/30/45/60 etc. min intervals). The user may adjust some or all; some may be bypassed selectively and can remain as dictated per program. The user may have the ability at any time to choose “back” to return to the previous option/screen. Once the user is finished adjusting parameters, the user can select start 475 and the training session can begin. The smart device can give audio and/or visual and/or vibratory, as applicable, instruction for the preparatory positioning correction alert 495 (e.g., preparatory posture correction, etc.) which can be of preset duration. Then, the training session can begin 500, again with audio and/or visual and/or vibratory instruction, as applicable, and repeat 505 until the number of repetitions or duration is complete at which time it can go into standby mode 510. Once the set time between sessions has elapsed 515, it can alert the user via reminder/notification of the next training session 405 as previously setup or auto-selected, or the user can manually begin at any time 410.

In regard to the various functions performed by the above described components, machines, devices, processes and the like, the terms (including a reference to a “means”) used to describe such components are intended to correspond, unless otherwise indicated, to any component which performs the specified function of the described component (e.g., a functional equivalent), even though not structurally equivalent to the disclosed structure, which performs the function in the herein illustrated exemplary aspects of the embodiments. In this regard, it will also be recognized that the embodiments include a system as well as electronic hardware configured to implement the functions, or a computer readable medium having computer-executable instructions for performing the acts or events of the various processes.

In addition, while a particular feature may have been disclosed with respect to only one of several implementations, such feature may be combined with one or more other features of the other implementations as may be desired and advantageous for any given or particular application. Furthermore, to the extent that the terms “includes,” and “including” and variants thereof are used in either the detailed description or the claims, these terms are intended to be inclusive in a manner similar to the term “comprising.” As used in this application, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

In other embodiments, combinations or sub-combinations of the above disclosed embodiments can be advantageously made. The block diagrams of the architecture and flow charts are grouped for ease of understanding. However, it should be understood that combinations of blocks, additions of new blocks, re-arrangement of blocks, and the like are contemplated in alternative embodiments of the present disclosure.

It is also understood that the examples and embodiments described herein are for illustrative purposes only and that various modifications or changes in light thereof will be suggested to persons skilled in the art and are to be included within the spirit and purview of this application and scope of the appended claims.

1. A device that facilitates an awareness and the activation of muscles of a user in a corrected posture, the device comprising:
one or more modules, wherein each module of the one or more modules is configured to be positioned adjacent to one or more associated muscles or regions of the user and each module of the one or modules comprises;
one or more alerting mechanisms, wherein each alerting mechanism of the one or more alerting mechanisms is configured to generate an associated alert to the user;
one or more attachment element(s) configured to attach the device to the user; and
a controller configured to operate the device.

2. The device according to claim 1, wherein a first alerting mechanism of the one or more alerting mechanisms comprises an electrically powered device producing one or more of a vibration, a visual output, an audible sound, a sensory level electrical stimulation, or a notification from a personal computer or smart device.

3. The device according to claim 1, wherein the one or more alerting mechanisms are configured to be activated sequentially, simultaneously, or separately.

4. The device according to claim 1, wherein the one or more muscles associated with a first module of the one or more modules are in a first muscle group that is distinct from a second muscle group comprising the one or more muscles associated with a second module of the one or more modules.

5. The device according to claim 1, wherein the one or more modules are configured to operate at least one of together or independently from one another.

6. The device according to claim 1, wherein said controller is configured to direct a first alerting mechanism of the one or more alerting mechanisms to issue its associated alert.

7. The device according to claim 1, further comprising a communication component configured to communicate with, receive communication from and store performance data from, one or more coupled devices, wherein each of the one or more coupled devices is one of a second device configured to operate specifically in connection with the device, a computer, or one or a smart device.

8. The device according to claim 16, further comprising a housing unit that comprises therewithin a printed circuit board (PCB), mechanism to communicate with the one or more coupled devices, a microswitch/timer, an electrically powered alerting mechanism, a battery, and a memory.

9. The device according to claim 1, wherein the one or more attachment elements comprise a securing mechanism with one or two faces of to secure the device to a user's skin in an intended area, wherein the securing mechanism is one or more of reusable, removable, or replaceable, wherein:
a first face of said securing mechanism is adhesive; and
a second face of said securing mechanism is one of: adhesive,
a first component of a hook and loop fastener,
or a second component of a hook and loop fastener.

10. The device according to claim 1, wherein said attachment element further comprises an article of clothing configured to assist in maintaining a positioning of the one or modules on the user when worn.

11. The device according to claim 9, wherein said attachment element comprises one of a clip, a snap, or a magnet, wherein the one of said clip, said snap or said magnet comprises two corresponding attachment components that facilitate attachment of a first component of the two corresponding attachment components to said one or more modules and that facilitate attachment of a second component of the two corresponding attachment components to said article of clothing.

12. The device according to claim 1, in which said one or more alerting mechanisms are configured to provide the associated alert to the user independently of said controller.

13. The device according to claim 1, further comprising at least one additional alerting mechanism comprising at least one of a computer or a smart device configured to generate one or more additional alerts for the user to correct a posture of the user and to activate at least a subset of the muscles one of with or without activation of said modules.

14. The device according to claim 1, wherein the one or more additional alerts comprise one or more of at least one image, at least one video, or at least one auditory instruction.

15. The device of claim 1 wherein a first module of the one or more modules comprises one or more sensors and one or more signal processors to detect muscle activation, muscle contraction, or posture.

16. A method for improving awareness and activation of muscles of a user in appropriate postures, comprising:
alerting a user to a correct posture via one or more modules, wherein each module of the one or more modules is configured to be positioned adjacent to one or more associated muscles or regions of the user; and
facilitating the activation of the muscles of a user of one or more intended regions of the body via generating one or more alerts via the one or more modules;
wherein the one or more alerts are generated one of sequentially or simultaneously.

17. The method of claim 16, wherein the one or more alerts are issued via at least one of a vibration, a visual output, an audible sound, a sensory level electrical stimulation or a notification from one of a computer or a smart device.

18. The method of claim 16 further comprising communicating data between the one or more modules and one or more coupled devices, wherein each of the one or more coupled devices is one of a second device configured to operate specifically in connection with the device, a computer, or a smart device.

19. The method of claim 18, wherein the data comprises performance data.

20. The method claim of 16, further comprising providing additional instruction to the user regarding muscle activation via one or more of: at least an images, at least one video, at least one interactive training, or at least one auditory instruction.

21. A method for facilitating the awareness and activation of one or more muscles in appropriate postures, comprising:
alerting a user to a correct posture via one or more of a computer or a smart device;
facilitating user activation of muscles of one or more regions of a body of the user via one or more alerts generated via the one or more of the computer or the smart device, wherein the one or more alerts facilitate one of sequential or simultaneous user activation of the muscles in a correct posture; and
the one or more alerts facilitate one of sequential or simultaneous user activation of the muscles in a correct posture via one or more of a computer and a smart device (smart phone, smart watch, portable device, e.g., tablet); and
providing instruction to the user to correct posture and activate one or more of at least one region of the body or the one or more muscles via one or more of a computer or a smart device.

22. The method of claim 21, wherein a first alert of the one or more alert comprises at least one of a vibration, a visual output, or an audible sound issued by one of a computer or a smart device.

23. The method of claim 21, further comprising providing to the user one or more of an application, a video, or an interactive training for correction of posture and activation of one or more intended regions of the body.

24. The method of claim 23, wherein the one or more of the application, the video, or the interactive training facilitate activation of the one or more intended regions of the body one of sequentially together, simultaneously together, or separately.

25. The method of claim 21, wherein the one or more alerts occur at repeated times throughout the day, with a frequency and a duration that is variable according to one or more of a preset program or based on user input.

26. The method of claim 21, wherein the one or more alerts facilitate training of one or more of the at least one region of the body and the one or more muscles one of together or independently.
